(19) United States
(12) Patent Application Publication
(10) Pub. No.: US 2012/0041235 A1
(43) Pub. Date: Feb. 16, 2012

(54) PROCESS FOR THE PREPARATION OF (R)-2-(3-DIISOPROPYLAMINO)-1-PHENYLPROPYL)-4METHYLPHENOL AND SALTS THEREOF

(75) Inventors: Theoharis V. Koftis, Thessaloniki (GR); Efstratios Neokosmidis, Thessaloniki (GR); Rohit Ravikant Soni, Thessaloniki (GR); Panagiota Mandalou, Thessaloniki (GR); Aristotelis Menisiou, Thessaloniki (GR)

(73) Assignee: PHARMATHEN S.A., PALLINI-ATTIKIS (GR)

(21) Appl. No.: 13/145,044

(22) PCT Filed: Feb. 17, 2009
(86) PCT No.: PCT/EP2009/001098

§ 371 Date: Jul. 18, 2011

(51) Int. Cl. C07C 213/02; C07B 57/00

(52) U.S. Cl. 564/304

(57) ABSTRACT

The present invention relates to an improved process for the preparation of Tolterodine or salts thereof, which comprises the use of 3-(2-methoxy-5-methylphenyl)-3-phenylpropyl methane sulfonate.

### TECHNICAL FIELD OF THE INVENTION

The present invention relates to an improved process for the preparation of (R)-2-(3-(diisopropylamino)-1-phenylpropyl)-4-methylphenol and pharmaceutical acceptable salts or derivatives thereof and in particular to a cost effective method for the preparation of (R)-Tolterodine L-tartrate (I) or salts thereof and pharmaceutical preparations containing said compounds.

### BACKGROUND OF THE INVENTION

Tolterodine is muscarinic receptor antagonist that reduces bladder hyperactivities in patients suffering from urinary incontinence. The therapeutic effect of tolterodine as well as its active metabolites in mammals is described in EP-B-667 852. The use of optically active tolterodine enantiomers in the treatment of urinary disorder is further described in WO03/002059.

Tolterodine and analogues thereof and methods for the preparation thereof, were first disclosed in EP-B-325 571. Said document discloses a process as summarized in Scheme 1. However, in said process reducing agent such as lithium aluminum hydride is used and the amination step takes place by heating the mixture of diisopropylamine and compound V in acetonitrile in a pressure bottle for 4˜6 days and the removing of the O-protective in compound VI lasts 2˜5 days'

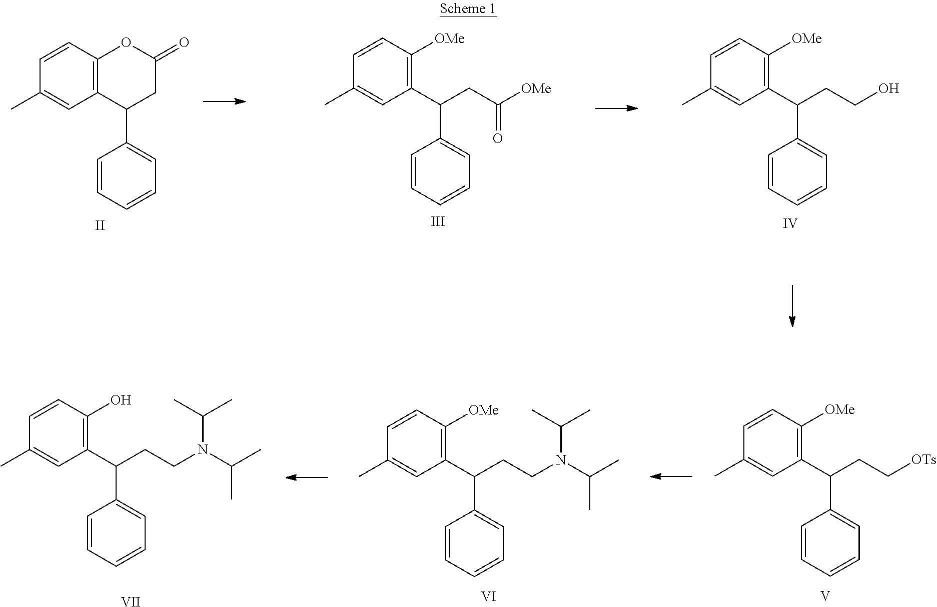

This procedure is a rather time-consuming process requiring hazardous and expensive materials as well as critical operating conditions. In addition to the long reaction time, the use of lithium aluminum hydride is not only highly corrosive and expensive, but it is also sensitive to moisture, air and heat, causing the operating conditions too demanding for commercial production, not to mention the low temperature requirement for the use of lithium aluminum hydride and the difficulties in quenching and removing the alumina residues from the reaction mass.

WO-A-2005/061432 discloses an process for the preparation of Tolterodine, wherein the deprotection of compound VI is carried out in the absence of solvent and preferably in the presence of pyridine hydrochloride under inert atmosphere however at elevated temperature, in the range of 200˜220° C., with the risk of forming hard mass that will make later process troublesome.

EP-B-960 109 discloses a process for the preparation of Tolterodine, through reductive animation of the respective lactone, derived from the coumarine derivativeII. This procedure requires the use of dibal-H at −20° C. to −25° C. for the reduction of the ketone and the reductive animation is performed by catalytic hydrogenation on palladium on carbon Pd/C. However, the same drawbacks exist as in the above mentioned prior art process, wherein lithium aluminum hydride is used.

Although each of the above patents represents an attempt to overcome the use of costly and hazardous material, there still exists a need for a cost-effective and safer process for commercial manufacture and less harmful to the personnel and environment.

### SUMMARY OF THE INVENTION

It is, therefore, an object of the present invention to provide an improved process for the preparation of Tolterodine or salts thereof or its derivatives, which overcomes the deficiencies of the prior art and results to a cost effective production without scarifying the yield and quality of the product.

Another object of the present invention is to provide an improved method for the preparation of Tolterodine or salts thereof, or its derivatives, by minimizing the presence of any contaminants and formed by-products during the reactions.

Another object of the present invention is to provide an improved method for the preparation of Tolterodine or salts thereof or its derivatives by selecting the appropriate reactants, solvents and catalysts used during the organic reactions, so that the purity and yield of reaction are increased.

In accordance with the above objects of the present invention, a process for the preparation of Tolterodine or pharmaceutically acceptable salts thereof or its derivatives is provided comprising the following steps:
(i) reacting 6-methyl-4-phenyl-3,4-dihydro coumarin (II) with methyl iodine (MeI) and potassium carbonate (K₂CO₃) to form methyl 3-(2-methoxy-5-methylphenyl)-3-phenylpropionate (III)

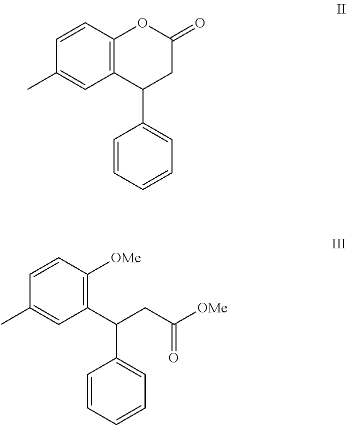

(ii) reducing the ester (III) to the corresponding alcohol 3-(2-methoxy-5-methylphenyl)-3-phenylpropanol (IV)

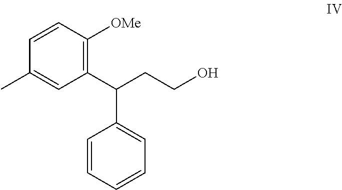

(iii) activating the hydroxyl group in compound of formula IV to the respective sulfonate intermediate (Vb)

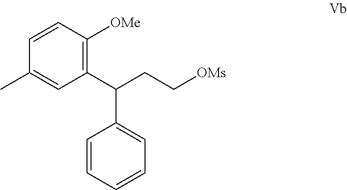

(iv) aminating with a mixture of diisopropylamine to form N,N-diisopropyl-3-(2-methoxy-5-methylphenyl)-3-phenylpropylamine and capturing the free base with hydrochloric acid to obtain crystalline salt (VIb)

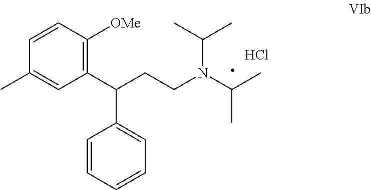

(v) removing the O-protective group to obtain racemic mixture of tolterodine (VII) and

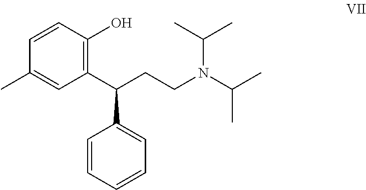

(vi) isolating the (R)-enantiomer from the racemic mixture of tolterodine by fractional crystallization of salts with chiral acid to obtain (R)-tolterodine L-tartrate (I).

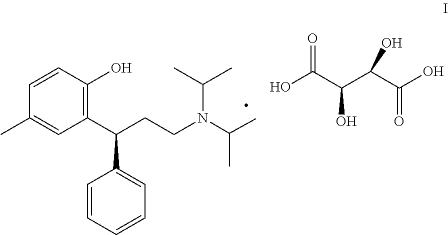

Preferred embodiments of the present invention are set out in dependent claims 2 to 6.

The ring-opening reaction in step (i) is carried out according to a known method with minor modifications, wherein methyl iodine (MeI) and potassium carbonate (K₂CO₃) are added in two batches.

The reducing agent used in step (ii) is sodium bis(2-methoxyethoxy)aluminum hydride (Red-Al). As a reagent, Red-Al is comparable with lithium aluminum hydride (LiAlH₄). In contrast with lithium aluminium hydride, Red-Al exhibits good solubility in aromatic solvents. Its solutions are also more stable to moisture and air than LiAlH₄ and more thermally stable, tolerating temperatures up to 200° C. Toluene solution of Red-Al is added drop wise to the toluene solution of compound III at low temperature and with proper workups, the reduction product 3-(2-methoxy-5-methylphenyl)-3-phenylpropanol (IV) can be obtained as a white to yellow thick oil.

The hydroxy protective moiety used in step (iii) is mesyl group. The reaction is performed in dichloromethane in the presence of triethylamine and after suitable workups, the sulfonate intermediate (Vb) can be obtained.

The amination in step (iv) is conducted by vigorously stirring the mixture of diisopropylamine, sodium iodide (Nap and compound Vb in acetonitrile in an autoclave. The volatile materials are evaporated with the aid of toluene and the free base is converted to its HCl salt (VIb), as the salt form is more stable.

The removal of the O-protective group can be achieved by treatment boron tribromide in dichloromethane, and after proper workups, racemic mixture of tolterodine can be received.

Resolution of the optical isomers can be achieved by converting the tolterodine hydrochloride salt to the corresponding tartrate salt. (R)-tolterodine L-tartrate (I) can be fractional crystallized out in methanol or ethanol.

Other objects and advantages of the present invention will become apparent to those skilled in the art in view of the following detailed description.

### DETAILED DESCRIPTION OF THE INVENTION

The present invention relates to an improved process for the preparation of Tolterodine and pharmaceutically acceptable salts thereof, which is characterized with substantially shorter reaction time, milder and safer reaction conditions without scarifying the yield and quality of the product and low cost of reactants and reagents.

According to the present invention, the process for the preparation of Tolterodine and pharmaceutically acceptable salts thereof, or its derivatives comprises the following steps:

### Preparation of 3-(2-methoxy-5-methylphenyl)-3-phenylpropionate

3-(2-methoxy-5-methylphenyl)-3-phenylpropionate of formula III is prepared according to a method as described in EP 325 571, Example 2, with minor modification, wherein certain amount of methyl iodine (MeI) and potassium carbonate (K₂CO₃) are mixed with 6-methyl-4-phenyl-3,4-dihydro coumarin (II) in methanol and heated at 55˜60° C. for approximately 4 h. A second batch of MeI and K₂CO₃ is added and the mass is re-heated at 55˜60° C. for additional 6 h until the reaction is completed. Subsequently the solvent is removed and the product is extracted by using toluene and water.

### Preparation of 3-(2-methoxy-5-methylphenyl)-3-phenylpropanol

3-(2-methoxy-5-methylphenyl)-3-phenylpropanol of formula IV is prepared by reducing 3-(2-methoxy-5-methylphenyl)-3-phenylpropionate of formula III using sodium bis(2-methoxyethoxy)aluminum hydride (Red-Al). Toluene solution of Red-Al is added slowly, over a period of at least 2 hours, to the toluene solution of compound III at temperature from about −5° C. to about 5° C. The resulting mixture is stirred at the same temperature until the reaction completes. Aqueous solution of NaOH is added drop wise to consume the excess Red-Al. The reaction mixture is filtered through a celite bed and the organic layer is washed with water and dry over anhydrous Na₂SO₄. After the removal of the toluene, compound of formula IV is obtained as a white to yellow thick oil.

### Preparation of 3-(2-methoxy-5-methylphenyl)-3-phenylpropyl methane sulfonate

3-(2-methoxy-5-methylphenyl)-3-phenylpropyl methane sulfonate of formula Vb is prepared by adding calculated amount of mesyl chloride (MsCl) slowly to the dichloromethane solution of compound IV in the presence of triethylamine at from about 0° C. to about 10° C. Then the reaction mixture is washed with water. The organic layer is separated and concentrated. Recrystallization is carried out in cyclohexane.

### Preparation of N,N-diisopropyl-3-(2-methoxy-5-methylphenyl)-3-phenylpropan-1-amine hydrochloride

3-(2-methoxy-5-methylphenyl)-3-phenylpropyl methane sulfonate of formula Vb is dissolved in acetonitrile and mixed with calculated amount of sodium iodide (NaI) and diisopropylamine. This solution is sealed in an autoclave reactor and vigorously stirred at about 100° C. After the reaction is completed, toluene is added and evaporated. This process is repeated to remove volatile materials. The remaining mass is extracted with water and the aqueous phase is back washed with toluene. The resulted N,N-diisopropyl-3-(2-methoxy-5-methylphenyl)-3-phenylpropan-1-amine free base is treated with hydrochloric acid. The crude material is recrystallized with toluene/cyclohexane mixture and compound of formula VI is received as light brown powder.

### Preparation of (R)-2-(3-(diisopropylamino)-1-phenylpropyl)-4-methylphenol L-tartrate

N,N-diisopropyl-3-(2-methoxy-5-methylphenyl)-3-phenylpropan-1-amie hydrochloride (VI) is dissolved in dichloromethane. Solution of BBr₃ is added drop wise under argon atmosphere. Neutralize the solution using NaOH and release the tolterodine free base. Chiral resolution of the optical isomers is conducted by adding a chrial resolution reagent, such as L-tartaric acid, and isolating the resulted salt by crystallization. (R)-2-(3-(diisopropylamino)-1-phenylpropyl)-4-methylphenol L-tartrate (I) is crystallized and re-crystallized in either methanol or ethanol.

### Example 1

### Preparation of 3-(2-methoxy-5-methylphenyl)-3-phenylpropyl methane sulfonate (Vb)

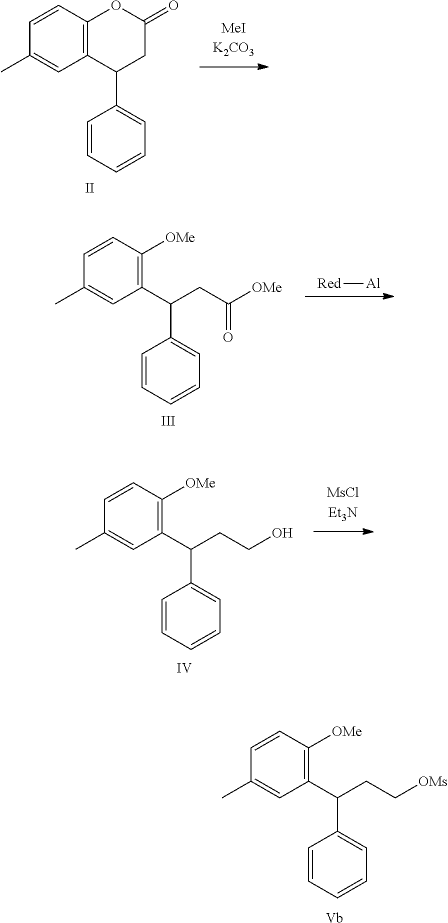

This intermediate is of significant importance for use in the preparation of Tolterodine or salts thereof, or its derivatives according to the present invention.

In a 3-necked round bottom flask, 6-methyl-4-phenyl-3,4-dihydro coumarin (II, 100 g, 0.420 mol) is charged into methanol (500 ml) at about 25° C. with stirring. To the resulting suspension, potassium carbonate (K₂CO₃, 87 g, 0.63 mol) and methyl iodide (MeI, 78 ml, 1.25 mol) are added and the mass is stirred at from about 55° C. to about 60° C. for 4 h. The reaction mass is cooled to from about 40° C. to about 45° C. and MeI (27 ml, 0.43 mol) and K₂CO₃ (29 g, 0.21 mol) are added. Re-heat the mass to 55˜60° C. and stir for additional 6 h until the reaction completes. Then solvent is removed by distillation under vacuum. The residue is dissolved in 350 ml of toluene. This organic phase is washed once with 750 ml of DM water and twice with 450 ml of DM water.

The organic layer thus obtained is charged into a 3-necked round bottom flask under N₂ atmosphere and cooled at temperature from about −5° C. to about 5° C. Toluene solution of sodium bis(2-methoxyethoxy) aluminum hydride (Red-Al, 200 ml, 70% w/v, 0.706 mol) is slowly added through a dropping funnel over a period of 120 min, maintaining the solution temperature within −5˜5° C. The mixture is stirred for an additional 1 h at this temperature until the reaction completes. Then aqueous solution of NaOH (80 ml, 10%) is added through a dropping funnel over a period of 45 min, maintaining the temperature at 0˜10° C., and continue stirring for an additional 45 min at the same temperature. Add 750 ml of DM water and stir for 30 min at 25° C. The biphasic reaction mass is filtered through a celite bed (10 g). The organic layer is collected and dried over 20 g of anhydrous sodium sulfate (Na₂SO₄). The solids are filtered and washed with 50 ml of toluene. The toluene is distilled off completely and compound IV is obtained as slightly yellow thick oil.

The thick oil is dissolved in 300 ml of dichloromethane (DCM) under N₂ atmosphere. Triethylamine (Et₃N, 90 ml, 0.647 mol) is added and the mass is cooled to 0˜5° C. Mesyl chloride (MsCl, 36 ml, 0.465 mol) is added slowly through a dropping funnel over a period of 120 min, keeping the solution temperature below 10° C. Continue stirring the mixture for an additional 1 h at 0˜5° C. until the reaction completes. 1 L of cold DM water (0˜5° C.) was added and the mass is stirred for 30˜60 min at this temperature. Conc. HCl (25 ml) is added and the mass is stirred until it reaches 25° C. The organic layer is collected and washed with 200 ml of DM water. About 180˜220 ml of DCM is distilled off from the organic layer under atmosphere pressure at 38˜43° C. 400 ml of cyclohexane is added to the residue and the mass is heated to reflux till a clear solution is obtained (70˜80° C.). This solution is cooled slowly and compound Vb crystallized. The solid is filtered in Buchner funnel and spray washed with 100 ml of cold cyclohexane. The mass is dried under vacuum at 60° C. to give 90˜110 g of title compound.

### Example 2

### Preparation of N,N-diisopropyl-3-(2-methoxy-5-methylphenyl)-3-phenylpropan-1-amie hydrochloride (VI)

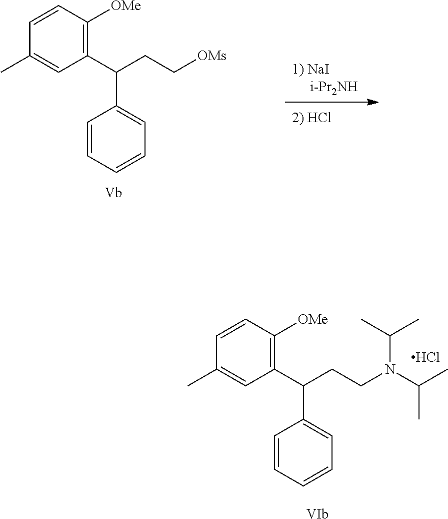

3-(2-Methoxy-5-methylphenyl)-3-phenylpropylmethane sulfonate (Vb, 100 g, 0.3 mol) was mixed with acetonitrile (700 ml) in an autoclave reactor. Sodium iodide (NaI, 112 g, 0.75 mol) and diisopropylamine (424 ml, 3.00 mol) are added to the solution. The reactor is sealed and the mass is heated to about 100° C. under vigorous stirring. After 4 hours the reaction is completed and 1 L of toluene is added to the reaction mass. Then it is evaporated until 1 L of solvent is collected. This procedure is repeated once again. The remaining mass is washed with 500 ml of DM water and the aqueous phase is washed with toluene (500 ml).

The organic layers are combined and dried over anhydrous Na₂SO₄ (20 g). The solid is filtered off and IPA·HCl (75 ml, 5˜6 N, 0.45 mol) is added drop wise to the filtrate under argon atmosphere. The mixture is stirred for 45˜60 min after the addition is completed. The mass is evaporated to dry. The crude material is re-crystallized with toluene/cyclohexane (400 ml/100 ml) and re-crystallized once more with toluene/cyclohexane (600 ml/150 ml). Light brown powder of N,N-diisopropyl-3-(2-methoxy-5-methylphenyl)-3-phenylpropan-1-amie hydrochloride (VI) is obtained in 85˜100 g.

### Example 3

### Preparation of crude (R)-2-(3-(diisopropylamino)-1-phenylpropyl)-4-methylphenol L-tartrate (I)

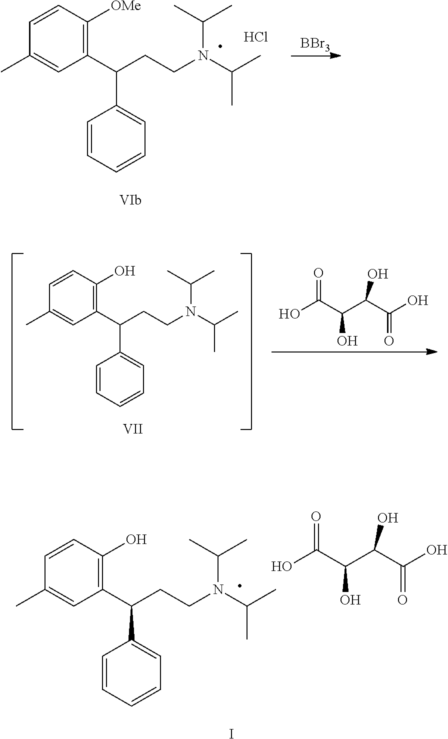

N,N-diisopropyl-3-(2-methoxy-5-methylphenyl)-3-phenylpropan-1-amie hydrochloride (VI, 100 g, 0.266 mol) is dissolved in dichloromethane (1100 ml) resulting in a clear solution. Under argon atmosphere, boron tribromide (BBr₃, 28 ml, 0.293 mol, 99%) is added drop wise. The solution is stirred for an additional 1 h. Aqueous solution of NaOH (680 ml, 10%) is added drop wisely at temperature from about 30° C. to about 35° C. and the mixture is stirred vigorously for 30 min. Separate the aqueous phase and extracted with dichloromethane (550 ml). The organic layers are combined and the solvents are evaporated. The corresponding hydroxylamine is obtained as brown viscous oil.

Methanol (300 ml) is added to the brown viscous oil and the mixture is heated until clear solution is achieved (40˜50° C.). To this solution, L-tartaric acid (44 g) is added and the mixture is refluxed for 2 h. Slowly cool the mixture to 5˜10° C. and stir for 1 h at this temperature. The solid is filtered and dried under vacuum for about 1 h. Then the solid is mixed with methanol (400˜450 ml) and heated to 60˜65° C. and stir until a clear solution is obtained. Filter this solution while hot to remove any non-soluble particles. Remove about 150 ml of methanol by distillation under atmosphere. The remaining suspension is gradually cooled down to 0˜5° C. and stir at this temperature for 1 h. Filter the suspension and dry the solid to yield 34˜39 g of title compound.

### Example 4

### Alternative Process for the Preparation of crude (R)-2-(3-(diisopropylamino)-1-phenylpropyl)-4-methylphenol L-tartrate (I)

N,N-diisopropyl-3-(2-methoxy-5-methylphenyl)-3-phenylpropan-1-amie hydrochloride (VI, 100 g) is dissolved in dichloromethane (1100 ml) resulting in a clear solution. Under argon atmosphere, boron tribromide (28 ml, 99%) is added drop wise. The solution is stirred for an additional 1 h. Aqueous solution of NaOH (680 ml, 10%) is added drop wise at temperature from about 30° C. to about 35° C. and the mixture is stirred vigorously for 30 min. Two phases are obtained and the aqueous phase is extracted with dichloromethane (550 ml). The organic layers are combined and the solvent is evaporated. The corresponding hydroxyl amine is obtained as brown viscous oil.

Ethanol (1000 ml) is added to the brown viscous oil and the mixture is stirred at 25˜30° C. until a clear solution is obtained. To this solution, a solution of L-tartaric acid (46 g) in ethanol (1000 ml) is added. The mixture is stirred for 30 min at 25˜30° C. Cool the mixture to 0˜5° C. and stir at this temperature for additional 3 h. The solid is filtered and dried under vacuum for about 1 h (dry under vacuum at 60° C. for 6 h). Then the solid is mixed with ethanol (ca. 1300˜1400 ml). The mixture is heated to 75˜80° C. with stirring until a clear solution is obtained. Filter this solution while hot to remove any non-soluble particles. Gradually cool down the filtrate to 0˜5° C. and stir at this temperature for 1 h. Filter the suspension and dry the solid to yield 45˜50 g of title compound as white powder.

### Example 5

### Purification of (R)-2-(3-(diisopropylamino)-1-phenylpropyl)-4-methylphenol L-tartrate (I)

Crude (R)-2-(3-(diisopropylamino)-1-phenylpropyl)-4-methylphenol L-tartrate (1.35 g) is mixed with methanol (315 ml) and heated to 60˜65° C. and stir until a clear solution is obtained. Remove 105 ml of methanol by distillation under atmosphere. The remaining suspension is cooled gradually to 0˜5° C. and stir this mixture for 1 h. Filter the suspension and dry the resulted solid to yield 26˜28 g of title compound as white powder.

### Example 6

### Alternative Process for the Purification of (R)-2-(3-(diisopropylamino)-1-phenylpropyl)-4-methylphenol L-tartrate (I)

Crude (R)-2-(3-(diisopropylamino)-1-phenylpropyl)-4-methylphenol L-tartrate (1.35 g) is mixed with ethanol (1400 ml) and heated to 75˜80° C. and stir until a clear solution is obtained. The solution is cooled gradually to 0˜5° C. and stir this mixture for 1 h. Filter the suspension under vacuum and dry the resulted solid to yield 28 g (80% w/w) of title compound as white powder.

Therefore, the present invention describes a method of preparing essentially pure Toletrodine and salts thereof in an improved manner.

While the present invention has been described with respect to the particular embodiments, it will be apparent to those skilled in the art that various changes and modifications may be made in the invention without departing from the spirit and scope thereof, as defined in the appended claims.

1. A process for preparation of tolterodine or pharmaceutically acceptable salts thereof or its derivatives, which comprises the following steps:
(i) reacting 6-methyl-4-phenyl-3,4-dihydro coumarin (II) with methyl iodine (MeI) and potassium carbonate (K₂CO₃) to form methyl 3-(2-methoxy-5-methylphenyl)-3-phenylpropionate (III) 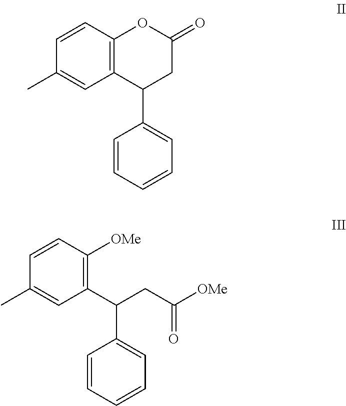
(ii) converting the ester (III) to the corresponding alcohol 3-(2-methoxy-5-methylphenyl)-3-phenylpropanol (IV) 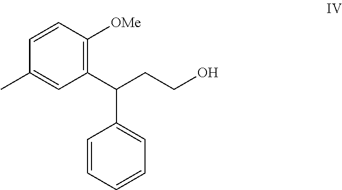
(iii) activating the hydroxyl group in compound IV to the respective sulfonate intermediate (Vb) 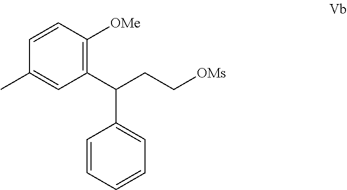
(iv) aminating with a mixture comprising diisopropylamine to form N,N-diisopropyl-3-(2-methoxy-5-methylphenyl)-3-phenylpropylamine and crystallization with hydrochloric acid to obtain the salt (VIb) 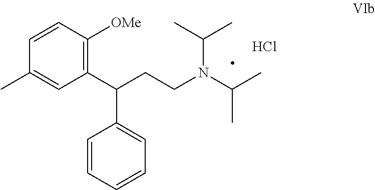
(v) removing the O-protective group to obtain racemic mixture of tolterodine (VII) and 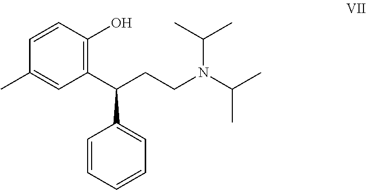
(vi) resolving the enantiomers to obtain (R)-tolterodine L-tartrate (I).

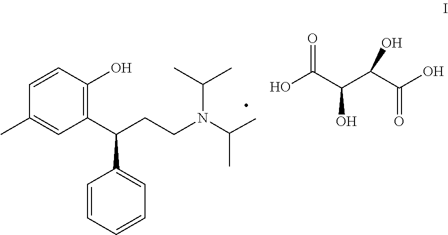

2. The process according to claim 1, wherein the reduction of step (ii) is carried out with sodium bis(2-methoxyethoxy)aluminum hydride, as a reducing agent.

3. The process according to claim 1, wherein the reaction of step (iii) is carried out in dichloromethane in the presence of triethylamine.

4. The process according to claim 1, wherein in step (iv) the solution of 3-(2-methoxy-5-methylphenyl)-3-phenylpropylmethane sulfonate (Vb) is reacted with a mixture of diisopropylamine and sodium iodide in one pot to obtain N,N-diisopropyl-3-(2-methoxy-5-methylphenyl)-3-phenylpropan-1-amine, and said reaction is carried out in an autoclave reactor at a temperature about 100° C. under vigorous stirring.

5. The process according to claim 1, wherein the removal of the O-protective group can be achieved by treatment boron tribromide in dichloromethane.

6. The process according to claim 1, wherein the resolution of the optical isomers can be achieved by converting the tolterodine hydrochloride salt to the corresponding tartrate salt and (R)-tolterodine L-tartrate (I) can be fractional crystallized out in methanol or ethanol.
